(19) United States
(12) Patent Application Publication
(10) Pub. No.: US 2008/0091057 A1
(43) Pub. Date: Apr. 17, 2008

(54) METHOD AND APPARATUS FOR PASSIVE LEFT ATRIAL SUPPORT

(75) Inventor: Joseph Walker, Shoreview, MN (US)

Correspondence Address: SCHWEGMAN, LUNDBERG & WOESSNER, P.A., P.O. BOX 2938, MINNEAPOLIS, MN, 55402, US

(73) Assignee: Cardiac Pacemakers, Inc., St. Paul, MN (US)

(21) Appl. No.: 11/548,394

(22) Filed: Oct. 11, 2006

(51) Int. Cl. A61F 2/00

(52) U.S. Cl. 600/37; 600/16

(57) ABSTRACT

One embodiment of the present subject matter includes a patch for attachment to a heart having a left atrium which includes a coronary sulcus, a line of reflection between the oblique and transverse pericardial sinuses, and left and right pulmonary veins, the patch sized to have a first edge portion disposed along the coronary sulcus and a second edge portion disposed along the line of reflection between the oblique and transverse pericardial sinuses. In one embodiment, the patch formed by the process including measuring left atrial diameter during over-distension, determining a patch size such that the patch is substantially inelastic when the left atrium is in over-distension, cutting the first edge portion of the patch to fit along the coronary sulcus and cutting the second edge portion of the patch to fit along the line of reflection between the oblique and transverse pericardial sinuses.

### TECHNICAL FIELD

This disclosure relates generally to implantable devices for the heart, and more specifically to method and apparatus for passive left atrial support.

### BACKGROUND

Left atrial failure potentially catalyzes the transition from diastolic dysfunction to heart failure with a normal ejection fraction (sometimes abbreviated as “HFnEF”). Left atrial failure often includes dilatation of the left atrium. Reduction or elimination of left atrial dilation could slow or prevent patients from progressing to heart failure with normal ejection fraction.

The left atrium is allowed to dilate in a natural state. It is desirable to allow the left atrium to fill in a passive state without allowing for over-distention. Any therapies which work to prevent over-distention should allow the left atrium to make a significant contribution during atrial systole. A solution which reduces negative effects arising from the left atrium being exposed to higher filling pressures associated with heart failure during normal ejection fraction is further desired.

### SUMMARY

The above-mentioned problems and others not expressly discussed herein are addressed by the present subject matter and will be understood by reading and studying this specification.

One embodiment of the present subject matter includes a patch for attachment to a heart having a left atrium which includes a coronary sulcus, a line of reflection between the oblique and transverse pericardial sinuses, and left and right pulmonary veins, the patch sized to have a first edge portion disposed along the coronary sulcus and a second edge portion disposed along the line of reflection between the oblique and transverse pericardial sinuses. In the embodiment, the patch is formed by the process of measuring left atrial diameter during over-distension, determining a patch size such that the patch is substantially inelastic when the left atrium is in over-distension, cutting the first edge portion of the patch to fit along the coronary sulcus and cutting the second edge portion of the patch to fit along the line of reflection between the oblique and transverse pericardial sinuses.

One embodiment of the present subject matter includes a method for reducing distention of a left atrium of a heart, the left atrium including a coronary sulcus and a line of reflection between the oblique and transverse pericardial sinuses. The embodiment includes covering the exterior of the left atrium with a patch, the covering of the exterior substantially limited to the left atrium, attaching a first edge portion of the patch to the heart proximal the coronary sulcus of the heart and attaching a second edge portion of the patch to the heart proximal the line of reflection between the oblique and transverse pericardial sinuses.

One embodiment of the present subject matter includes an apparatus for attachment to a left atrium of a heart. The apparatus includes a patch for providing support to the left atrium such that the left atrium does not distend during left atrium diastole and a connection apparatus for connecting the patch means to the heart.

This Summary is an overview of some of the teachings of the present application and not intended to be an exclusive or exhaustive treatment of the present subject matter. Further details about the present subject matter are found in the detailed description and appended claims. Other aspects will be apparent to persons skilled in the art upon reading and understanding the following detailed description and viewing the drawings that form a part thereof, each of which are not to be taken in a limiting sense. The scope of the present invention is defined by the appended claims and their legal equivalents.

### BRIEF DESCRIPTION OF THE DRAWINGS

FIG. 1 illustrates a heart with a patch, according to one embodiment of the present subject matter.

FIG. 2 illustrates a heart with a patch, according to one embodiment of the present subject matter.

### DETAILED DESCRIPTION

The following detailed description of the present subject matter refers to subject matter in the accompanying drawings which show, by way of illustration, specific aspects and embodiments in which the present subject matter may be practiced. These embodiments are described in sufficient detail to enable those skilled in the art to practice the present subject matter. References to “an”, “one”, or “various” embodiments in this disclosure are not necessarily to the same embodiment, and such references contemplate more than one embodiment. The following detailed description is demonstrative and not to be taken in a limiting sense. The scope of the present subject matter is defined by the appended claims, along with the full scope of legal equivalents to which such claims are entitled.

Various embodiments of the present subject matter provide a support which attaches to the exterior of the heart at least partially along the left atrium. In various embodiments, the support restricts dilation of the left atrium during left ventricular filling. Embodiments of the present subject matter prevent the left atrium from being undesirably distended. Embodiments of the present subject matter additionally allow for the left atrium to make a significant contribution during atrial systole. In various embodiments, the support allows the left atrium to accommodate higher filling pressures associated with heart failure with a normal ejection fraction. Various embodiments of the present subject matter allow filling of the left atrium during HFnEF while reducing instances of decompensation events.

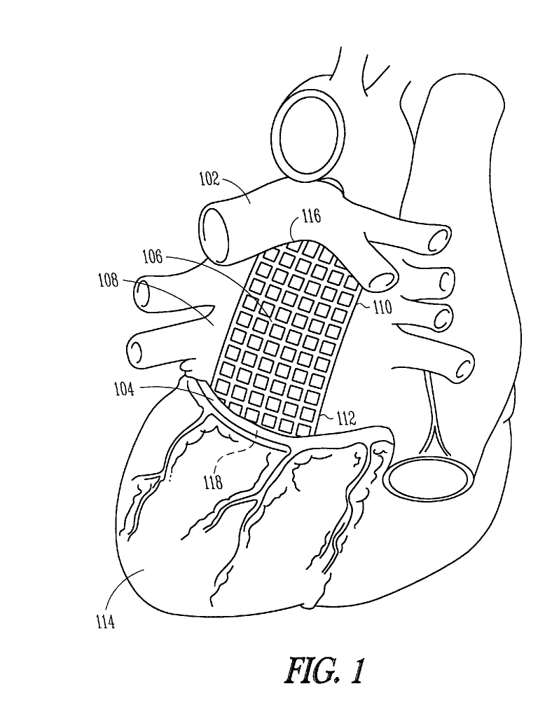

FIG. 1 illustrates a heart 114 with a patch 106, according to one embodiment of the present subject matter. Various embodiments of the present subject matter provide a patch 106 for attachment to a heart 114, and/or to tissues located proximal the heart 114. In various embodiments, the patch 106 is positioned outside the epicardium and inside the pericardium. In various embodiments, a patch 106 includes, but is not limited to, one or more of the following constructions: nets, meshes, screens and/or sheets. In various embodiments, a patch 106 defines one or more grooves which are adapted to accommodate blood vessels. In some embodiments, a patch 106 includes features, including, but not limited to, grooves, holes, anchors, and other features, which may be integrated with a lead. Among the embodiments contemplated by the present subject matter are cardiac pacing leads. Some patch 106 embodiments are used in combination with other heart therapies. In various embodiments, a patch includes one or more openings permits passage of electrical contacts through the patch. In some of these embodiments, the electrical contacts are part of a lead. Heart therapies used in conjunction with the present subject matter include, but are not limit to, electrical therapies such as pacing, defibrillation, cardioversion, and other electrical therapies, drug therapies, and/or other therapies not expressly recited herein. In various embodiments, integrating patch 106 with an electrical therapy can decrease energy consumption during electrical therapy. This can result in lower energy shocks, which can improve patient comfort. Lower energy shocks can additionally prolong device lifespan, leading to fewer device servicing events. Lower energy shocks can additionally provide for a smaller device. Integrating a lead with a patch 106 can additionally result in improved placement of the lead with respect to the heart 114.

In various embodiments, the patch 106 is semipermeable. In some embodiments, the whole patch 106 can be made semipermeable or totally permeable depending upon the patch 106 size and physiological requirements. In some embodiments, the patch 106 is perforated to have numerous holes. Substantially continuous sheets are contemplated. Some of these sheets do not include apertures defined by the sheet.

In various embodiments, the patch 106 includes sutures which are used to attach the patch 106 to the heart 114. In some embodiments, the patch 106 includes features to which sutures can attach. Some embodiments include eyelets, for example. Other anchors are additionally contemplated. For example, in some embodiments, a patch 106 attached to a heart 114 with adhesive. In various embodiments one or more sutures are attached on one side or both sides of the patch 106. Suturing makes the patch 106 fit tightly onto the heart 114. Some embodiments use a purse string suture to adjust fit of the patch 106 to the heart 114.

In various embodiments, the patch 106 is sutured to the heart 114 at a first position 110 and a second position 112. In various embodiments, the first position 110 is located proximal the left atrium 108 and the pulmonary artery 102. In various embodiments, the attachment occurs along the line of reflection 116 between the oblique and transverse pericardial sinuses. In various embodiments, the second position 112 is located proximal the left atrium 108 and proximal the coronary sinus 104. In various embodiments, the second position 112 is along the coronary sulcus 118.

In various embodiments, suture locations may be spaced along the first and second positions. In various embodiments, a care provider may select sutures along the first and second location so that the patch 106 is secure and conforms to left atrium 108. In various embodiments, a surgeon can add additional suture locations along the patch 106 to reduce shifting of the patch 106 after placement. In various embodiments, the number of suture locations is selected such that the patch 106 does not restrict contraction of the heart 114 during systole. In various embodiments, the patch 106 is adapted to be substantially inflexible when stretched between the first portion and the second portion. Such a configuration reduces instances of left atrium over-distention, in various embodiments.

Various materials are contemplated for use in a patch 106 of the present subject matter. In addition to materials listed herein elsewhere, embodiments contemplated include, but are not limited to, one or more of the following materials: polyetherurethane, polycarbonateurethane, peroxide cure silicone, high density polyethylene, platinum cured silicone, silicone, polysiloxaneurethane, polyfluoroethylene, or hydrogenated poly(styrene-butadiene) copolymer, poly(siloxane) urethane, ethylene-propylene, dicyclopentadiene terpolymer, hydrogenated poly(styrene-butadiene) copolymer, poly(tetramethylene-ether glycol) urethanes, poly(hexamethylenecarbonate-ethylenecarbonate glycol) urethanes. In various embodiments, materials are chosen based on to their elasticity. In some embodiments, materials are selected based on their biocompatibility.

In various embodiments, the patch 106 is radioopaque. In various embodiments, this improves the ability of a care provider to view the left atrium using radiography. In various embodiments, the patch 106 provides and improved definition of the left atrium when the left atrium is viewed with radiography. In various embodiments, this improves the ability of the care provider to diagnosis diseases which afflict the left atrium.

In various embodiments, the patch 106 is prepared by casting, coating, extruding, molding of these biocompatible and biocompatible materials. These processes are not an exhaustive or exclusive list of the processes contemplated by the present subject matter, and additional processes are contemplated. In various embodiments, a patient's heart is measured and recorded, and recorded data is used to perform a patch 106 which is customized to fit a patient. For example, in some embodiments, an MRI is used to determine the shape of a patient's heart, and then the MRI data is used to develop a patch for the patient. In additional embodiments, an echocardiogram is used to determine the size of the heart.

In various embodiments, the patch 106 is reinforced with fibers. In various embodiments, fibers demonstrate improved tensile strength. For example, in some embodiments, a patch 106 demonstrates improved tensile strength when connected to fibers. In example of such a configuration includes a sheet interwoven intermittently with fibers. In some embodiments, the patch 106 consists exclusively of a weave of fibers. In addition to fibers made from materials described herein, fibers, in various embodiments, include, but are not limited to, the following materials: polyamide, polyester, and polypropylene or crosslinked polyurethane, poly(ethylene terephthalate), and/or poly(butylene terephthalate).

Fibers which are used in various embodiments include reinforcing filaments having tensile strengths which vary from filament to filament. The patch 106 of this invention can be reinforced with mono-filaments yarns, braids, cords, knitted, woven and/or non-woven cloth. In various embodiments, in addition to materials listed herein elsewhere, materials for a cloth include, but are not limited to, polyimide, polyester, polypropylene, and/or polyurethane.

In various embodiments, the patch 106 is constructed from a knit, biocompatible material. In some embodiments, the knit is an Atlas knit. In various embodiments which include an Atlas knit, fibers of the knit have directional expansion properties. In some of these embodiments, the knit is formed of inelastic fibers which permit a construction of a flexible fabric which has limited expansion. For example, in some embodiments, a patch 106 including a knit of the present subject matter allows the left atrium 108 to expand, but not into over-distention.

The knit material, in various embodiments, is flexible and permits desired movements of the heart 114. In various embodiments, the material is open and defines a plurality of interstitial spaces. In various embodiments, such spaces provide for fluid permeability. Spaces may be selected in the interest of reducing the amount of surface area in direct contact with the heart 114. In some embodiments, this reduces scarring, including, but not limited to, fibrosis.

In various embodiments, the open areas of the knit construction allow for electrical connection between the heart and surrounding tissue for passage of electrical current to and from the heart. For example, although in some embodiments the knit material is an electrical insulator, a construction including openings allows for a patient to receive external defibrillation.

Various embodiments include a patch 106 which is flexible. Embodiments contemplated by the present subject matter affix a patch 106 to a heart 114 under elastic tension, so that the patch 106 is put into tension as the left atrium 108 relaxes, and stretches as the left atrium 108 relaxes. Some of these embodiments restrict expansion substantially before over-distention occurs. Additional embodiments are contemplated in which a patch 106 is not under tension when affixed to the heart 114, and is not put under tension until the left atrium 108 attempts to stretch substantially into over-distention. In some of these embodiments, a weave is selected which allows for elastic conformance of the patch 106 to the left atrium 108 during normal heart 114 activity, and which substantially prevents unwanted over-distention of the left atrium 108. In some embodiments, a patch including a chain-like structure is used in order to provide for slack before over-distension commences, and to provide a rigid structure after over-distention begins. Other weaves are contemplated. Some embodiments include a bunched weave which is elastic while bunched and which becomes substantially inelastic once the material is stretched so that it is no longer bunched.

In some embodiments, heart size is measured, and a patch is selected which has slack before over-distention, and does not expand after over-distension begins. In some of these embodiments, over-distension commences on or around a left atrial diameter of approximately 5 centimeters.

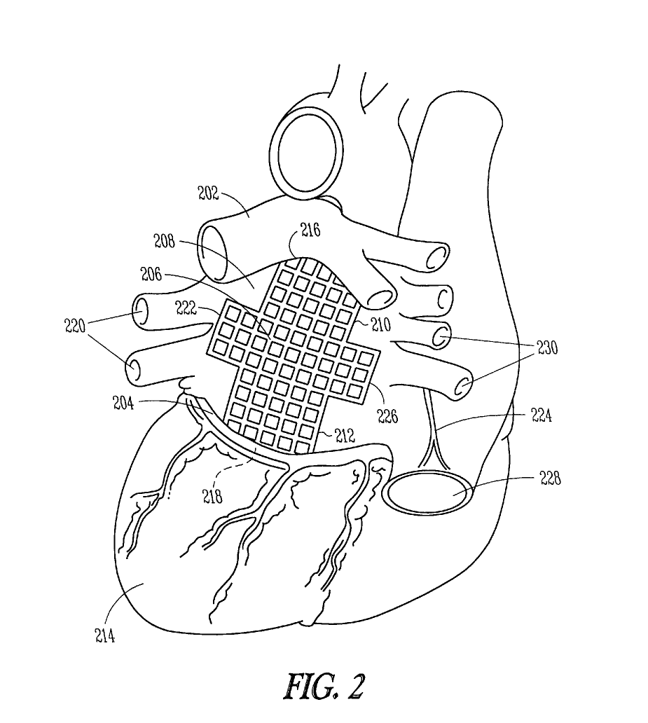

FIG. 2 illustrates a heart with a left atrial support, according to one embodiment of the present subject matter. In various embodiments, the patch 206 is secured to the heart 214 through sutures. In various embodiments, the patch 206 is sutured to the heart 214 at a first position 210 and a second position 212. In various embodiments, the first position 210 is located proximal the left atrium 208 and the pulmonary artery 202. In various embodiments, the attachment occurs along the line of reflection between the oblique and transverse pericardial sinuses 216. In various embodiments, the second position 212 is located proximal the left atrium 208 and proximal the coronary sinus 204. In various embodiments, the second position 212 is along the coronary sulcus 218.

In various embodiments, the patch 206 is secured to the heart 214 through sutures. In various embodiments, the patch 206 is sutured to the heart 214 at a third position 222 and a fourth position 226. In various embodiments, the third position 222 is located proximal the left atrium 208 and the left pulmonary veins 220. In various embodiments, the fourth position 226 is located proximal the left atrium 208 and proximal the interatrial groove 224. In various embodiments, the fourth portion 226 attaches proximal the inferior vena cava 228. In various embodiments, the fourth portion 226 attaches proximal the right pulmonary veins 230. In various embodiments, the fourth portion 226 attaches to the heart 214 along tissue extending between the inferior vena cava 228 and the right pulmonary veins 230. In various embodiments, the patch 206 is adapted to be substantially inflexible when stretched between the third portion and the fourth portion.

The present subject matter includes various methods for reducing left atrial distention. Some embodiments of the present subject matter include surgically accessing a patient's heart. In some of these methods, the left atrium is covered with a patch. In some embodiments, a flexible patch is attached to the heart. In some embodiment, the covering of the exterior of the heart is substantially limited to covering the left atrium. In various embodiments, the patch is attached with sutures. In some embodiments, the patch is attached with adhesive. In some embodiments, the patch is first sutured to the heart, and then further suturing the patch around the left atrium so that the patch is snug to the left atrium during left atrial relaxation during normal heart function. In some of these embodiments, the patch is sutured to itself. In various embodiments, the patch is adjusted such that the patch substantially prevents distention of the left atrium during atrial filling. In various embodiments, the patch is adjusted so that there is slack in the patch during contraction.

The present subject matter includes patches for attachment to a heart having a left atrium which includes a coronary sulcus, a line of reflection between the oblique and transverse pericardial sinuses, and left and right pulmonary veins. Some patches of the present subject matter are sized to have a first edge portion disposed along the coronary sulcus. Some patches of the present subject matter are sized to have a second edge portion disposed along the line of reflection between the oblique and transverse pericardial sinuses. Some patches are sized to have a third edge portion of the patch to fit along a left atrium proximal at least one left pulmonary vein. In some of these embodiments, the patch is sized to have an edge which extends to the junction of multiple left pulmonary veins. In some of these embodiments, the patch extends around this junction. Some embodiments of the present subject matter include a patch which includes a fourth edge portion of the patch to fit along at least one right pulmonary vein and the interatrial groove. In some of these embodiments, the patch is sized to have an edge which extends to the junction of multiple right pulmonary veins. In some of these embodiments, the patch extends around this junction. Some embodiments extend along the interatrial groove.

In various embodiments, the embodiment, the patch is formed by the process of measuring left atrial diameter during over-distension. In some embodiments, the left atrial diameter which signals over-distension is approximately 5 centimeters. In various embodiments, the present subject matter includes determining a patch size such that the patch is substantially inelastic when the left atrium is in over-distension. In various embodiments, the present subject matter includes cutting the first edge portion of the patch to fit along the coronary sulcus. Various embodiments additionally include cutting the second edge portion of the patch to fit along the line of reflection between the oblique and transverse pericardial sinuses. Embodiments of the present subject matter additionally include cutting the third edge portion of the patch to fit along the left pulmonary vein. Various embodiments additionally include cutting the fourth edge portion of the patch to fit proximal the right pulmonary vein. In some of these embodiments, the patch fits along the interatrial groove.

Although specific embodiments have been illustrated and described herein, it will be appreciated by those of ordinary skill in the art that any arrangement which is calculated to achieve the same purpose may be substituted for the specific embodiment shown. This application is intended to cover adaptations or variations of the present subject matter. It is to be understood that the above description is intended to be illustrative, and not restrictive. Combinations of the above embodiments, and other embodiments will be apparent to those of skill in the art upon reviewing the above description. The scope of the present subject matter should be determined with reference to the appended claims, along with the full scope of equivalents to which such claims are entitled.

What is claimed is:

1. A patch for attachment to a heart having a left atrium which includes a coronary sulcus, a line of reflection between the oblique and transverse pericardial sinuses, and left and right pulmonary veins, the patch sized to have a first edge portion disposed along the coronary sulcus and a second edge portion disposed along the line of reflection between the oblique and transverse pericardial sinuses, the patch formed by the process comprising:
measuring left atrial diameter during over-distension;
determining a patch size such that the patch is substantially inelastic when the left atrium is in over-distension;
cutting the first edge portion of the patch to fit along the coronary sulcus; and
cutting the second edge portion of the patch to fit along the line of reflection between the oblique and transverse pericardial sinuses.

2. The patch by the process of claim 1, further comprising suturing the patch to itself to remove slack in the patch.

3. The patch by the process of claim 1, further comprising a plurality of eyelets along at least the first edge portion.

4. The patch by the process of claim 1, wherein over-distension occurs when the left atrium diameter is greater than approximately 5 centimeters.

5. The patch by the process of claim 1, further comprising:
cutting a third edge portion of the patch to fit along a left atrium proximal at least one left pulmonary vein; and
cutting a fourth edge portion of the patch to fit along at least one right pulmonary vein and the interatrial groove.

6. The patch by the process of claim 1, wherein the patch includes at least one polymer.

7. The patch by the process of claim 6, wherein the patch includes silicon.

8. The patch by the process of claim 6, wherein the patch includes polyfluoroethylene.

9. A method for reducing distention of a left atrium of a heart, the left atrium including a coronary sulcus and a line of reflection between the oblique and transverse pericardial sinuses, the method comprising:
covering the exterior of the left atrium with a patch, the covering of the exterior substantially limited to the left atrium;
attaching a first edge portion of the patch to the heart proximal the coronary sulcus of the heart; and
attaching a second edge portion of the patch to the heart proximal the line of reflection between the oblique and transverse pericardial sinuses.

10. The method of claim 9, further comprising suturing the patch to the heart.

11. The method of claim 9, further comprising determining if normal ejection fraction exists for the heart.

12. The method of claim 11, further comprising determining if heart failure exists.

13. The method of claim 9, further comprising folding the patch unto itself and attaching the patch to itself to cinch the patch against the left atrium.

14. The method of claim 13, further comprising suturing the patch to itself.

15. The method of claim 9, further comprising:
attaching a third edge portion of the patch to fit along a left atrium proximal at least one left pulmonary vein; and
attaching a fourth edge portion of the patch to fit along at least one right pulmonary vein and the interatrial groove.

16. The method of claim 15, further comprising suturing the third edge portion and the fourth edge portion to the heart.

17. An apparatus for attachment to a left atrium of a heart, the apparatus comprising:
patch means for providing support to the left atrium such that the left atrium does not distend during left atrium diastole; and
connection means for connecting the patch means to the heart.

18. The apparatus of claim 17, wherein the connection means include at least one suture.

19. The apparatus of claim 17, wherein the patch means include a flexible patch connected to the heart at a first connection proximal the left atrium and proximal a coronary sulcus of the heart and at a second connection proximal the left atrium and proximal an interatrial groove of the heart and extending between the first connection and the second connection.

20. The apparatus of claim 19, wherein the flexible patch includes a mesh.
